(19) United States
(12) Patent Application Publication
(10) Pub. No.: US 2008/0167548 A1
(43) Pub. Date: Jul. 10, 2008

(54) Tissue Alteration With MRI RF Field

(76) Inventor: Alma G. Sorensen, Belmont, MA (US)

Correspondence Address: QUARLES & BRADY LLP, 411 E. WISCONSIN AVENUE, SUITE 2040, MILWAUKEE, WI, 53202-4497, US

(21) Appl. No.: 11/969,478

(22) Filed: Jan. 4, 2008

Related U.S. Application Data
(60) Provisional application No. 60/878,619, filed on Jan. 4, 2007.

(51) Int. Cl. A61B 5/055; G01R 33/32; A61N 2/02

(52) U.S. Cl. 600/411; 324/318; 600/10

(57) ABSTRACT

A system and method for altering a selective area of tissue in a patient uses an MRI system including a coil array having a plurality of separate coil elements. Each coil element is coupled to a respective one of the RF transmitters. The method includes identifying a target tissue area in a patient and selecting a pulse sequence configured to produce a prescribed RF field at target tissue area to heat the target tissue area. An agent is administered to the patient that is configured to enhance a medical procedure and the pulse sequence is applied to the patient using the coil elements to heat the target tissue area sufficiently to assist the agent with enhancing the medical procedure. The method further includes performing a medical imaging process to at least monitor heating of the target tissue area.

### BACKGROUND OF THE INVENTION

This application is based on U.S. Provisional Patent Application Ser. No. 60/878,619 filed on Jan. 4, 2007 and entitled “TISSUE ALTERATION WITH MRI RF FIELD”.

### BACKGROUND OF THE INVENTION

The field of the invention is nuclear magnetic resonance imaging (MRI) methods and systems. More particularly, the invention relates to RF coil systems for applying an excitation field to a subject under MRI examination to alter tissues.

When a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, Mz, may be rotated, or “tipped”, into the x-y plane to produce a net transverse magnetic moment Mt. A signal is emitted by the excited spins after the excitation signal B₁ is terminated, this signal may be received and processed to form an image.

When utilizing these signals to produce images, magnetic field gradients (Gₓ, G_{y} and G_{z}) are employed. Typically, the region to be imaged is scanned by a sequence of measurement cycles in which these gradients vary according to the particular localization method being used. The resulting set of received NMR signals are digitized and processed to reconstruct the image using one of many well known reconstruction techniques.

In recent years RF coils have been developed that include multiple coil elements and that enable the acquisition of multiple NMR signals in parallel. The use of such parallel receive channels enables a number of parallel processing methods such as SENSE and SMASH to be used that shorten the total scan time for acquiring an MR image.

These same coil elements can be driven by separate RF transmitters to produce highly tailored RF excitation fields. As described in published US Patent Application No. 2004/0199070, these coil elements can also be driven by the RF transmitters to produce focused RF energy for hyperthermic treatment of tissues. The MRI system may then acquire NMR image data that enables a temperature map of the target region to be produced.

However,

### SUMMARY OF THE INVENTION

The present invention is a method and system for altering target tissues during a medical procedure. Specifically, an MRI system is used to deliver focused RF energy to alter target tissues during the medical procedure. The application of focused RF energy and acquisition of MR data using the MRI system may be performed in interleaved fashion to monitor and control the medical procedure in real time.

In accordance with one aspect of the invention, focused RF energy is produced during a brain imaging processing using an MRI system and a contrast agent. The focused RF energy is controlled to deliver sufficient energy to break down the blood-brain barrier and enable the MRI contrast agent to flow into brain tissues. An image is then acquired of the brain tissues that are perfused with the contrast agent.

In accordance with still another aspect of the invention, focused RF energy is produced during brain imaging to break down the blood-brain barrier sufficiently to enable a chemotherapeutic agent or other compound to flow into brain tissues. This is followed by acquiring an MR image of the same blood vessel to ascertain the result of the alteration.

In accordance with another aspect of the invention, a blood clot in a particular blood vessel is identified within a region of interest using an MRI system. A thrombolysis agent is then administered and the MRI system is used to focus RF energy at the target blood clot in a blood vessel. The process is monitored in real time using the MRI system to determine proper deposition of energy and effectiveness of the therapy.

Various other features of the present invention will be made apparent from the following detailed description and the drawings.

### BRIEF DESCRIPTION OF THE DRAWINGS

FIG. 1 is a block diagram of an MRI system configured for use with the present invention;

FIG. 2 is a block diagram of a transceiver of the MRI system of FIG. 1;

FIG. 3 is a flow chart setting forth the steps for performing one procedure in accordance with the present invention;

FIG. 4 is a flow chart setting forth the steps for performing another procedure in accordance with the present invention; and

FIG. 5 is a perspective view of a coil array for use with the transceiver of FIG. 2.

### DESCRIPTION OF THE PREFERRED EMBODIMENT

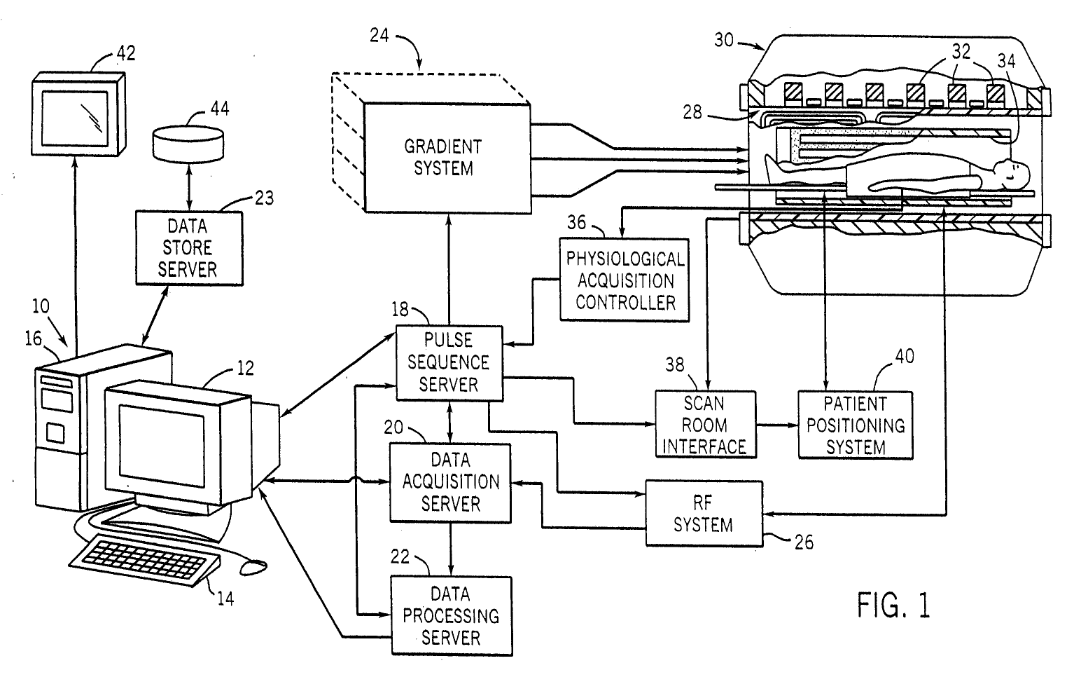

Referring particularly to FIG. 1, the preferred embodiment of the invention is employed in an MRI system manufactured by Siemens Medical Solution of Erlangen, Germany. The MRI system includes a workstation 10 having a display 12 and a keyboard 14. The workstation 10 includes a processor 16 that is a commercially available programmable machine running a commercially available operating system. The workstation 10 provides the operator interface that enables scan prescriptions to be entered into the MRI system. The workstation 10 is coupled to four servers: a pulse sequence server 18; a data acquisition server 20; a data processing server 22, and a data store server 23.

The pulse sequence server 18 functions in response to program elements downloaded from the workstation 10 to operate a gradient system 24 and an RF system 26. Gradient waveforms necessary to perform the prescribed scan are produced and applied to the gradient system 24 that excites gradient coils in an assembly 28 to produce the magnetic field gradients Gₓ, G_{y} and G_{z} used for position encoding NMR signals. The gradient coil assembly 28 forms part of a magnet assembly 30 that includes a polarizing magnet 32 and a whole-body RF coil 34.

RF excitation waveforms are applied to the RF coil 34 by the RF system 26 to perform the prescribed magnetic resonance pulse sequence. Responsive NMR signals detected by a separate RF coil array described below are received by the RF system 26, amplified, demodulated, filtered and digitized under direction of commands produced by the pulse sequence server 18. The RF system 26 includes an RF transmitter for producing a wide variety of RF pulses used in MR pulse sequences. The RF transmitter is responsive to the scan prescription and direction from the pulse sequence server 18 to produce RF pulses of the desired frequency, phase and pulse amplitude waveform.

The RF system 26 also includes a plurality of RF receiver channels. In the preferred embodiment 90 receiver channels are employed. Each RF receiver channel includes an RF amplifier that amplifies the NMR signal received by the coil to which it is connected and a quadrature detector that detects and digitizes the I and Q quadrature components of the received NMR signal. The magnitude of the received NMR signal may thus be determined at any sampled point by the square root of the sum of the squares of the I and Q components:

M=\sqrt{I²+Q²}

and the phase of the received NMR signal may also be determined:

Φ=tan⁻¹Q/I

The pulse sequence server 18 also optionally receives patient data from a physiological acquisition controller 36. The controller 36 receives signals from a number of different sensors connected to the patient, such as ECG signals from electrodes or respiratory signals from a bellows. Such signals are typically used by the pulse sequence server 18 to synchronize, or “gate”, the performance of the scan with the subject's respiration or heart beat.

The pulse sequence server 18 also connects to a scan room interface circuit 38 that receives signals from various sensors associated with the condition of the patient and the magnet system. It is also through the scan room interface circuit 38 that a patient positioning system 40 receives commands to move the patient to desired positions during the scan.

The digitized NMR signal samples produced by the RF system 26 are received by the data acquisition server 20. The data acquisition server 20 operates to receive the real-time NMR data and provide buffer storage such that no data is lost by data overrun. In some scans the data acquisition server 20 does little more than pass the acquired NMR data to the data processor server 22. However, in scans that require information derived from acquired NMR data to control the further performance of the scan, the data acquisition server 20 is programmed to produce such information and convey it to the pulse sequence server 18. For example, during prescans NMR data is acquired and used to calibrate the pulse sequence performed by the pulse sequence server 18. Also, navigator signals may be acquired during a scan and used to adjust RF or gradient system operating parameters or to control the view order in which k-space is sampled. And, the data acquisition server 20 may be employed to process NMR signals used to detect the arrival of contrast agent in an MRA scan. In all these examples the data acquisition server 20 acquires NMR data and processes it in real-time to produce information that is used to control the scan.

The data processing server 22 receives NMR data from the data acquisition server 20 and processes it in accordance with an image reconstruction method. Images reconstructed by the data processing server 22 are conveyed back to the workstation 10 where they are stored. Real-time images may be output to operator display 12 or a display 42 that is located near the magnet assembly 30 for use by attending physicians. Batch mode images or selected real time images are stored in a host database on disc storage 44. The workstation 10 may be used by an operator to archive the images, produce films, or send the images via a network to other facilities.

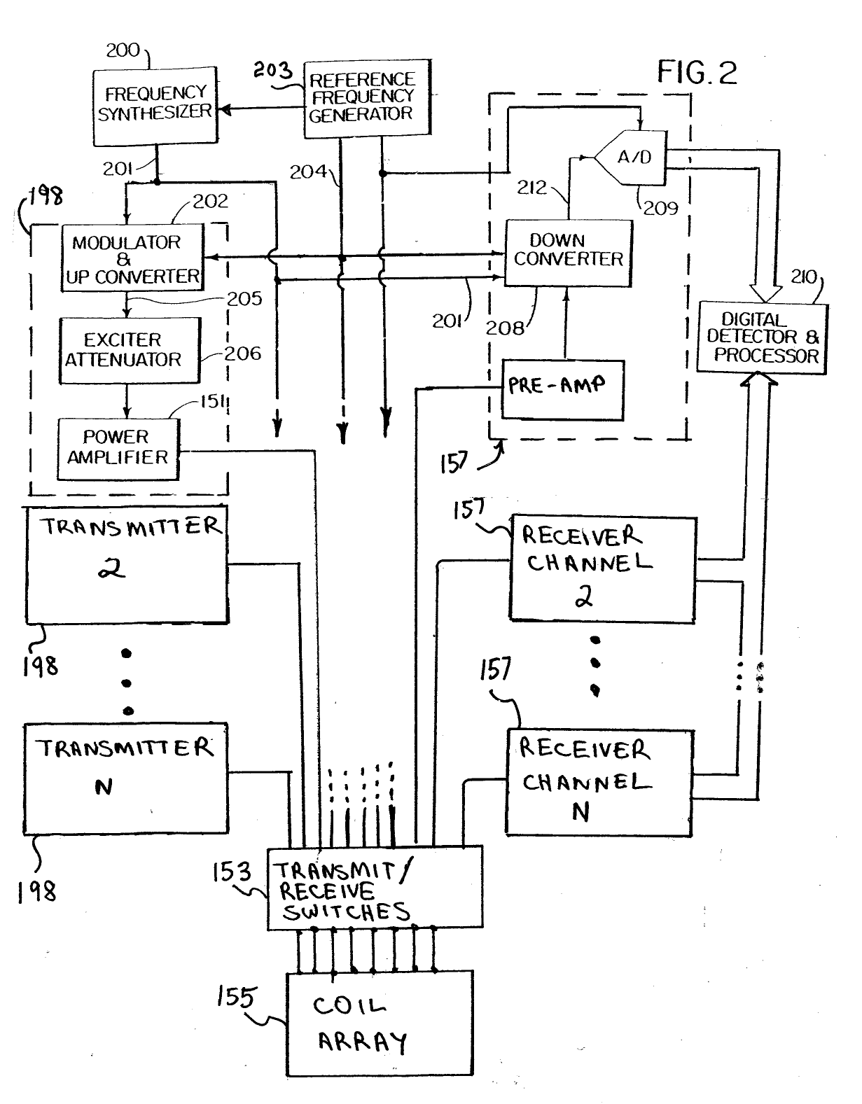

Referring particularly to FIG. 2, the RF system 26 includes a set of transmitters 198 that each produce a prescribed RF excitation field. The base, or carrier, frequency of this RF excitation field is produced under control of a frequency synthesizer 200 that receives a set of digital signals from the pulse sequence server 18. These digital signals indicate the frequency and phase of the RF carrier signal produced at an output 201. The RF carrier is applied to a modulator and up converter 202 in each transmitter 198 where its amplitude is modulated in response to a signal R(t) also received from the pulse sequence server 18. The signal R(t) defines the envelope of the RF excitation pulse to be produced and is produced by sequentially reading out a series of stored digital values. These stored digital values may, be changed to enable any desired RF pulse envelope to be produced by each transmitter 198.

The magnitude of the RF excitation pulse produced at output 205 is attenuated by an exciter attenuator circuit 206 in each transmitter that receives a digital command from the pulse sequence server 18. The attenuated RF excitation pulses are applied to a power amplifier 151 in each transmitter 198. The power amplifiers are current source devices that connect to respective transmit inputs on a set of transmit/receive switches 153. N transmitters 198 are employed and connected through N transmit/receive switches 153 to N coil elements in a coil array 155.

Referring still to FIG. 2 the signal produced by the subject is picked up by the coil array 155 and applied to the inputs of a set of receive channels 157. A pre-amplifier 160 in each receiver cannel 157 amplifies the signal by an amount determined by a digital attenuation signal received from the pulse sequence server 18. The received signal is at or around the Larmor frequency, and this high frequency signal is down converted in a two step process by a down converter 208 that first mixes the NMR signal with the carrier signal on line 201 and then mixes the resulting difference signal with a reference signal on line 204. The down converted NMR signal is applied to the input of an analog-to-digital (A/D) converter 209 that samples and digitizes the analog signal and applies it to a digital detector and signal processor 210 that produces 16-bit in-phase (I) values and 16-bit quadrature (Q) values corresponding to the received signal. The resulting stream of digitized I and Q values of the received signal are output to the data acquisition server 20. The reference signal as well as the sampling signal applied to the A/D converter 209 are produced by a reference frequency generator 203.

The transmit/receive switches 153 are operated by the pulse sequence server 18 to connect the N transmitters 198 to the N coil elements in the coil array 155 during those parts of the pulse sequence in which an RF field is to be produced. Each transmitter 198 is separately controlled by the pulse sequence server 18 to produce an RF field of a prescribed amplitude, frequency, phase and envelope at each of the N coil elements. The combined RF fields of the N coil elements produce the prescribed B₁ field throughout the region of interest in the subject during the imaging phase of the procedure. These same coil elements are used during the tissue alteration phase of the procedure to focus the RF energy at prescribed target tissues as described in more detail below.

When the B₁ field is not produced the pulse sequence server 18 operates the transmit/receive switches 153 to connect each of the N receive channels to the respective N coil elements. Signals produced by excited spins in the subject are picked up and separately processed as described above.

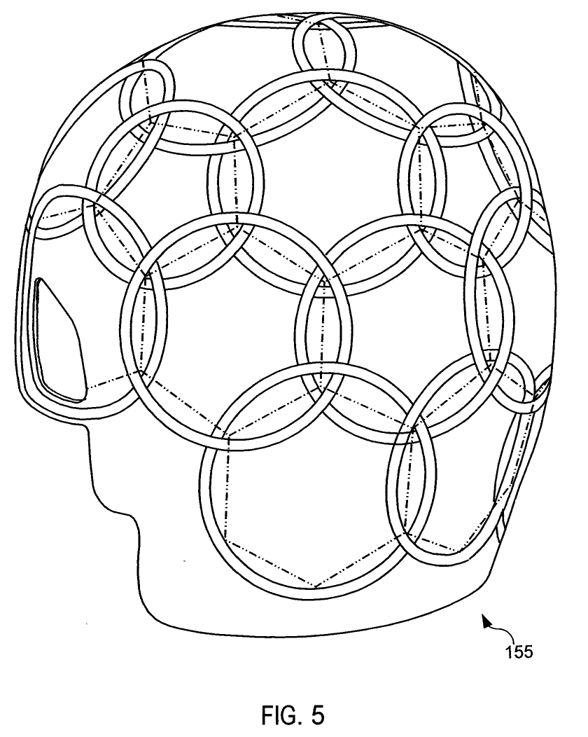

The coil array 155 is shown in FIG. 5. The close-fitting fiberglass helmet structure of the array 155 is modeled after the European head standard from EN960/1994 for protective headgear. This coil array 155 has 90 separate RF coil elements that are positioned over the curved helmet surface. Each coil element is substantially circular in shape and adjacent coil elements overlap such that their mutual inductance is minimized. As described in co-pending PCT application WO 2005/109010A2 filed on May 3, 2005 and entitled “Coil Array Positioning”, inductive coupling between coil elements is reduced by overlapping adjacent coil elements and using preamplifier decoupling. The cable leading from each of the 90 coil elements through the transmit/receive switch to the preamplifier in its corresponding receiver channel is carefully chosen and the tuning of the matching circuit to the preamplifier is chosen to transform the high preamplifier input impedance to a low impedance across the circular coil element. An arrangement of hexagonal and pentagonal tiles cover the helmet surface, similar to a geodesic tiling of a sphere. Each tile has sides that are approximately 23 mm long although it was necessary to distort the pentagonal tiles in places in order to map them onto the surface of the helmet. A circular surface coil is centered on each one of the tiles. Each surface coil is made from 0.031 inch thick G10 copper clad circuit board with a conductor width of 2.5 mm. The diameter of each coil element ranges from 4.5 cm to 5.5 cm. It has been found that significant 5 to 8-fold gains in SNR are possible with this structure as compared to conventional head coils, particularly in the cerebral cortex.

Distributing the coil elements all over the surface of the head provides greater flexibility in controlling the B₁ field created by the coil, either through adjustment of the phase and amplitude of the RF signal sent to each element, or through the use of different coil elements over the surface of the head allows acceleration in any chosen direction. The soccer-ball-type geometries and the principle of arranging the elements close to the head with an even spatial distribution in all directions improves B₁ shimming and enables generation of focused RF energy regardless of the design of the individual elements and the decoupling methods used. In addition, liquid nitrogen may be employed to enable superconducting-like behavior in the coil elements. This improves the efficiency of the coil during RF transmission.

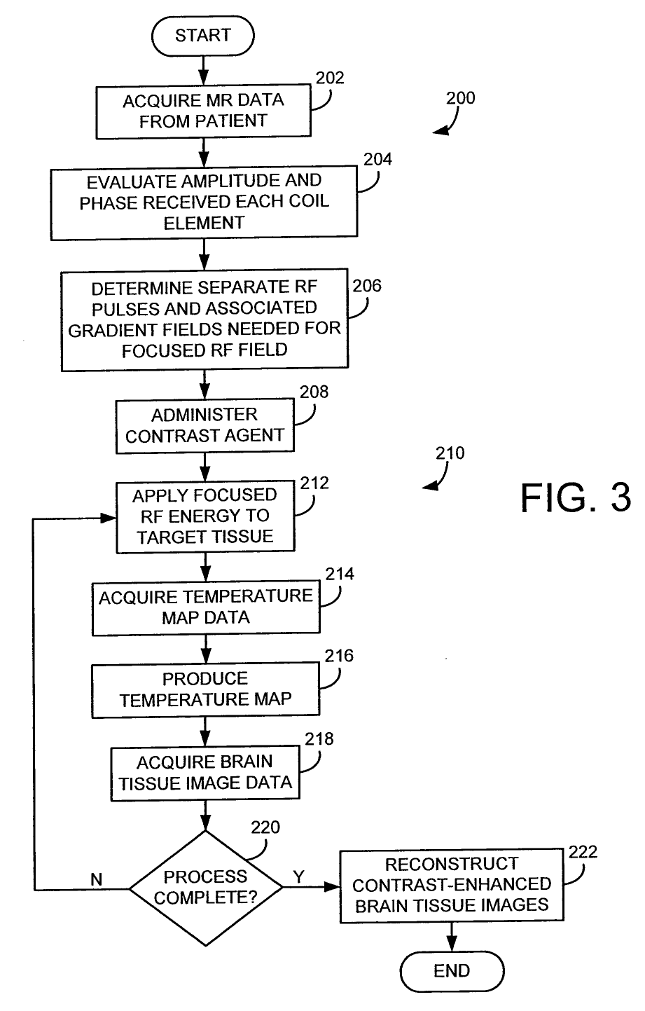

Referring particularly to FIG. 3, one method in accordance with the present invention uses the focused RF energy produced by the coil array 155 to break down the blood-brain barrier. In this clinical application, the blood-brain barrier is broken only momentarily to allow contrast agent to perfuse into tissues surrounding the target blood vessels. However, as will be described, the delivery of RF energy to accomplish this task without causing permanent damage must be precisely controlled.

To specifically target the region where the blood-brain barrier is to be broken down, a scout scan is performed, as generally indicated at process block 200. This scout scan functions as a tuning scan in which the magnetic resonance signals received from a body region at process block 202 are evaluated at process block 204 in terms of their amplitude and phase received at individual coil elements. This tuning sequence is in the form of an free induction decay (FID) measurement with suitable actuation of gradient field coils to prevent the emission of NMR signals from regions of the body that are not of interest. As a result of the scout scan, the separate RF pulses and associated gradient fields are produced at process block 206 such that an RF field focused on the target tissues can be produced.

Thereafter, a contrast agent is administered, as indicated at process block 208, and a loop 210 is entered in which the focused RF energy is applied to the target tissues at process block 212. During the application of the focused RF energy the RF transmitters 198 described above with respect to FIG. 5 produce the prescribed RF pulses determined by the scout scan to generate the controlled RF energy at the target tissues. This energy is controlled to be sufficient to disrupt the blood-brain barrier for a short period of time. To monitor the effectiveness of the RF energy and to ensure that a minimized amount of RF energy needed to disrupt the blood-brain barrier is applied, temperature map data is acquired at process block 214 from which a temperature map is created at process block 216. Once it is identified from the temperature maps that sufficient energy has been delivered to the region of interest to disrupt the blood-brain barrier, whereby the contrast agent in the blood leaks into the surrounding brain tissue to provide the desired tissue contrast. Accordingly, brain tissue image data is acquired at process block 214. The imaging pulse sequence, such as a three-dimensional, fast, gradient-recalled echo pulse sequence, takes advantage of this contrast mechanism to provide images with enhanced brain tissue contrast.

Typically, image data is acquired over a period of time during which the contrast agent arrives at the imaging region of interest and perfuses into surrounding tissues. When the dynamic study is complete as determined at decision block 220, the scan is complete and the series of acquired images is reconstructed as indicated at process block 222.

A variation of this method may be used to disrupt the blood-brain barrier and, instead of delivering a contrast agent, a chemotherapeutic agent may be delivered into the surrounding brain tissue. Image data may be acquired in this variation to confirm that the therapeutic agent has penetrated to the desired region of the brain.

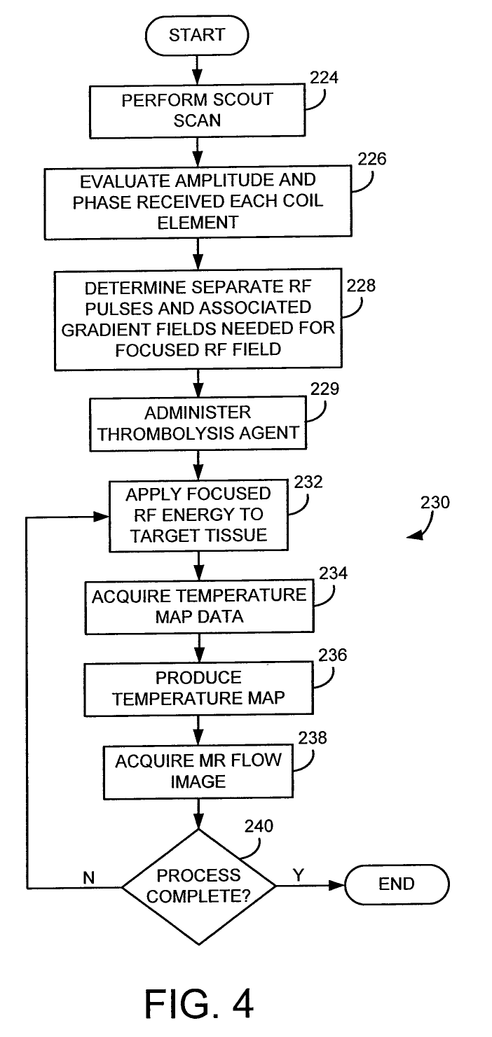

Another clinical application of the present invention is to perform thrombolysis on a blood clot located in a blood vessel. In this application, the focused RF energy disrupts the clot to assist in a traditional thrombolysis procedure and enable blood to more freely flow through the blood vessel. In particular, referring to FIG. 4, the first step in this thrombolysis treatment process is to conduct a scout scan, as indicated at process block 224, in which the blood clot and surrounding area are imaged. As described above, based on the results of the scout scan, the RF pulses produced by each RF transmitter 198 and the gradient fields applied are tailored at process block 226 to excite the blood clot. From this, a prescription for producing the desired focused energy at the blood clot is determined at process bock 228. A thrombolysis agent is administered at process block 229. For example, the thrombolysis agent may include agents such as streptokinase, urokinase, alteplase, reteplase, tenecteplase, and the like.

A loop 230 is then entered in which the blood clot is radiated with the focused RF energy, as indicated at process block 232. To monitor the effectiveness of the RF energy and minimize the amount of RF energy applied, temperature map data is acquired at process block 234 from which a temperature map is created at process block 236. In a manner similar to that described above when targeting the blood-brain barrier, the RF energy is precisely focused on the blood clot and its energy is controlled to disrupt the blood clot without damaging surrounding tissues. Certain parts of the clot may be targeted (such as the most distal or most proximal to the parent vessel) to more safely or effectively allow for clot disruption and to allow the thrombolysis agent improved access to the clot.

During this iterative process, the surrounding region is imaged, as indicated at process block 238. In one embodiment, the MR image is velocity encoded with a bipolar gradient oriented along the direction of the central axis of the blood vessel. The resulting reconstructed image indicates blood velocity through the blood vessel at the blood clot location. The process of irradiating the blood clot and acquiring an MR image continues until the operator sees adequate blood flow through the clotted blood vessel as determined at decision block 240. The procedure is then terminated.

Therefore, a system and method is described for altering target tissues during a medical procedure. Specifically, an MRI system is used to deliver focused RF energy to alter target tissues during the medical procedure. The application of focused RF energy and acquisition of MR data using the MRI system may be performed in interleaved fashion to monitor and control the medical procedure in real time.

The present invention has been described in terms of the various embodiments, and it should be appreciated that many equivalents, alternatives, variations, and modifications, aside from those expressly stated, are possible and within the scope of the invention. Therefore, the invention should not be limited to a particular described embodiment.

1. A method of altering a selective area of tissue in a patient using an MRI system including a coil array having a plurality of separate coil elements, each coil element coupled to a respective one of the RF transmitters, the method comprising:
a) identifying a target tissue area in a patient;
b) selecting a pulse sequence configured to produce a prescribed RF field at target tissue area to heat the target tissue area;
c) administering an agent to the patient configured to enhance a medical procedure;
d) applying the pulse sequence using the coil elements to heat the target tissue area sufficiently to assist the agent with enhancing the medical procedure; and
e) performing a medical imaging process to at least monitor heating of the target tissue area.

2. The method of claim 1 wherein the agent includes a contrast agent and wherein the target tissue area includes the blood-brain barrier.

3. The method of claim 2 wherein step d) includes heating the blood-brain barrier to alter the permeability of the blood-brain barrier to permit the contrast agent to cross the blood-brain barrier.

4. The method of claim 3 wherein step e) includes acquiring an image of a brain of the patient after the contrast agent has crossed the blood-brain barrier.

5. The method of claim 1 wherein the agent includes a thrombolytic agent and the target tissue area includes a blood clot.

6. In an MRI system comprising:
a plurality of separately controllable RF transmitters;
a coil array having a plurality of separate coil elements, each coil element coupled to a respective one of the RF transmitters and the coil elements being positioned to surround an anatomical region of interest; and
a pulse generator operable to separately control the plurality of transmitters such that the coil array produces a prescribed RF field in the anatomical region of interest that alters tissues therein, and operable to produce a prescribed B₁ RF field in the anatomical regions of interest during the acquisition of an MR image.

7. The MRI system of claim 6 wherein the altered tissue is in the brain of a subject and the alteration breaks the blood-brain barrier.

8. The MRI system of claim 6 wherein the altered tissue is a blood vessel containing a blood clot and the alteration breaks up the blood clot to enable improved blood flow through the blood vessel.

9. The MRI system of claim 6 wherein a contrast agent is injected into the subject.

10. The MRI system of claim 6 wherein a therapeutic agent is injected in the subject.
